(19) United States
(12) Patent Application Publication
(10) Pub. No.: US 2002/0131997 A1
(43) Pub. Date: Sep. 19, 2002

(54) Method for treating hematologic disorders with water insoluble 20 (S)-camptothecin

(76) Inventors: Hagop M. Kartarjian, Bellaire, TX (US); Beppino C. Giovanella, Houston, TX (US); Ethan A. Natelson, Houston, TX (US)

Correspondence Address: LUKE A. KILYK, ESQ., KILYK & BOWERSOX, P.L.L.C., 3603-E CHAIN BRIDGE ROAD, FAIRFAX, VA, 22030, US

(21) Appl. No.: 09/556,020

(22) Filed: Apr. 20, 2000

(51) Int. Cl. A61F 13/00; A61K 9/20

(52) U.S. Cl. 424/464; 424/465

(57) ABSTRACT

A method is provided for treating a patient afflicted with a hematologic disorder such as chronic leukemia and the myelodysplastic syndromes. The method includes administering to the patient an effective amount of a water-insoluble 20(S)-camptothecin compound with a closed lactone ring, a derivative thereof, or a mixture thereof. In a preferred method, the compound administered is 9-nitro-20(S)-camptothecin. The compound can be administered orally, intramuscularly, transdermally, subcutaneously, or parenterally.

### FIELD OF THE INVENTION

The present invention relates to methods of treating hematologic disorders, and, in particular, to methods of treating the myelodysplastic syndromes and the use of particular compositions for the treatment.

### BACKGROUND OF THE INVENTION

Hematologic disorders include abnormal growth of blood cells which can lead to dysplastic changes in blood cells and hematologic malignancies such as various leukemias.

Acute myeloid leukemia (AML) is the most common type of acute leukemia that occurs in adults. Several inherited genetic disorders and immunodeficiency states are associated with an increased risk of AML. These include disorders with defects in DNA stability, leading to random chormosomal breakage, such as Bloom's syndrome, Fanconi's anemia, Li-Fraumeni kindreds, ataxia-telangiectasia, and X-linked agammaglobulinemia. Cytoarabine (Ara-C) has been used alone or in combination with anthracycline or daunorubicin to treat AML.

Acute promyelocytic leukemia (APML) represents a distinct subgroup of AML. This subtype is characterized by promyelocytic blasts containing the 15;17 chromosomal translocation. This translocation leads to the generation of the fusion transcript comprised of the retinoic acid receptor and a sequence PML. Retinoic acid and its derivatives have been used to treat APML and have been shown to induce differentiation of blasts in patients with APML.

Acute lymphoblastic leukemia (ALL) is a heterogenerous disease with distinct clinical features displayed by various subtypes. Reoccurring cytogenetic abnormalities have been demonstrated in ALL. The most common cytogenetic abnormality is the 9;22 translocation. The resultant Philadelphia chromosome represents poor prognosis of the patient. Vincristine, anthracyclines, and prednisone have been used to treat ALL.

Chronic myelogenous leukemia (CML) is a clonal myeloproliferative disorder of a pluripotent stem cell. CML is characterized by a specific chromosomal abnormality involving the translocation of chromosomes 9 and 22, creating the Philadelphia chromosome. Ionizing radiation is associated with the development of CML. Hydroxyurea, interferon (INF) and Ara-C have been used to treat patients with CML.

The myelodysplastic syndromes (MDS) are heterogeneous clonal hematopoietic stem cell disorders grouped together because of the presence of dysplastic changes in one or more of the hematopoietic lineages including dysplastic changes in the myeloid, erythroid, and megakaryocytic series. These changes result in cytopenias in one or more of the three lineages. Patients afflicted with MDS typically develop complications related to anemia, neutropenia (infections), or thrombocytopenia (bleeding). Generally, from about 10% to about 70% of patients with MDS develop acute leukemia.

The prognosis of patients with MDS is generally poor with a median survival range of from about 6 months to about 2 years, depending upon the French-American-British (FAB) subset and karyotype. Patients with MDS die either from complications associated with the disease transformation to acute leukemia, or while in the MDS phase from infections and bleeding.

Patients with MDS are categorized by the FAB classification as follows: (1) refractory anemia (RA); (2) refractory anemia with ringed sideroblasts (RAS); (3) refractory anemia with excess blasts (RAEB); and (4) refractory anemia with excess blasts in transformation (RAEB-t). MDS is described in Heaney et al, “myelodysplasia”, New Eng J of Med, Vol. 340, pp 1649-60 (1999) and Hofman et al, “Current Therapeutic Options in Myelodysplastic Syndromes”, Hematology, Vol. 4, pp 91-112 (1999), which are incorporated herein in their entireties by reference. High-risk MDS is associated with a median survival of 1 year or less. Despite research efforts, there are no agents approved for the treatment of MDS.

Therapies for the treatment of MDS have included (1) supportive care, (2) biological agents (interferons), (3) growth factors (erythropoietin, G-CSF, GM-CSF), (4) intensive chemotherapy, (5) allogeneic bone marrow transplantation (BMT), and (6) low dose chemotherapy or differentiating agents (low-dose ara-C, azacytidine). Growth factors may improve cytopenias but do not induce complete remissions (CR), and may not prolong survival. Allogeneic BMT applies to a minority of patients and is a high-risk but curative procedure. Intensive chemotherapy induces complete remissions in 40% to 50% of patients, but is associated with serious morbidity and mortality and requires repetitive and prolonged periods of hospitalization with consequent impairment of the quality of life. Remissions following intensive chemotherapy are short-lasting, and survival is not prolonged. Consequently, it remains an investigational modality which is toxic, and without proven benefit to patients. Low-dose ara-C and azacytidine have induced responses in about 25% to 50% of patients with the exception of allogeneic BMT, none of these approaches clearly improves patient prognosis. None of these approaches induce favorable responses in a majority of patients.

Although some success has been found using topotecan chemotherapy in the treatment of MDS, the treatment is intensive, aggressive, and intravenous and thus is not accepted by a wide patient pool. Furthermore, there is no salvage options for patients who relapse after camptothecin-based chemotherapy. A need exists for a safe and effective method for treating the myelodysplastic syndromes.

### SUMMARY OF THE PRESENT INVENTION

The present invention provides methods for treating hematologic disorders, in particular, chronic leukemia and the myelodysplastic syndromes using water insoluble 20(S)-camtothecin compounds.

In one aspect of the present invention, a method is provided for treating treating a hematologic disorder in a patient by using water insoluble 20(S)-camtothecin compound in oral dosage forms. The method comprises: orally administering into the patient suffering from a hematologic disorder a composition that is adapted for oral administration and comprises an effective amount of water-insoluble 20(S)-camptothecin compound with a closed-lactone ring.

According to this method the water-insoluble 20(S)-camptothecin compound is 20(S)-camptothecin, an analog of 20(S)-camptothecin, or a derivative of 20(S)-camptothecin. The analogs and derivatives of 20(S)-camptothecin also include pharmaceutically active metabolites of 20(S)-camptothecin and prodrugs of 20(S)-camptothecin. In a preferred embodiment, the water-insoluble 20(S)-camptothecin compound is 9-nitro-20(S)-camptothecin, 9-amino-20(S)-camptothecin, or combinations thereof.

Also according to this method, the water-insoluble 20(S)-camptothecin compound is administered orally to a patient at an effective amount preferably from about 0.5 mg/m² to about 5.0 mg/m² of body surface area of the patient daily, more preferably from about 1.0 mg/m² to about 3.0 mg/m² of body surface area of the patient daily, and most preferably from about 1.5 mg/m² to about 2.5 mg/m² of body surface area of the patient daily.

Also according to this method, the water-insoluble 20(S)-camptothecin compound is administered orally to a patient at an effective amount daily for preferably 3-6 days a week and more preferably 4-5 days a week.

This method can be used to treat a hematologic disorder such as various forms of leukemia. Forms or classes of leukemia include, but are not limited to, chronic myeloid leukemia (CML), chronic myelomonocytic leukemia (CMML), acute myeloid leukemia (AML), and acute lymphoblastic leukemia (ALL). In particular, this method can be used to treat a patient with CML, Philadelphia chromosome (Ph)-negative or Ph-positive CML.

In another aspect of the present invention, a method is provided for treating the myelodysplastic syndrome (MDS) using a water-insoluble 20(S)-camptothecin compound with a closed-lactone ring. The method comprises: administering to said patient a composition comprising an effective amount of water-insoluble 20(S)-camptothecin compound of the closed lactone ring form.

According to this method, the water-insoluble 20(S)-camptothecin compound is 20(S)-camptothecin, an analog of 20(S)-camptothecin, or a derivative of 20(S)-camptothecin. The analogs and derivatives of 20(S)-camptothecin also include pharmaceutically active metabolites of 20(S)-camptothecin and prodrugs of 20(S)-camptothecin. In a preferred embodiment, the water-insoluble 20(S)-camptothecin compound is 9-nitro-20(S)-camptothecin, 9-amino-20(S)-camptothecin, or combinations thereof.

Also according to this method, the composition comprising water-insoluble 20(S)-camptothecin compound is orally administered into a human patient with MDS. The effective amount for oral administration is ranging preferably from about 0.5 mg/m² to about 5.0 mg/m² of body surface area of the patient daily, more preferably from about 1.0 mg/m² to about 3.0 mg/m² of body surface area of the patient daily, and most preferably from about 1.5 mg/m² to about 2.5 mg/m² of body surface area of the patient daily.

Also according to this method, the water-insoluble 20(S)-camptothecin compound is administered orally to a MDS patient at an effective amount daily for preferably 3-6 days a week and more preferably 4-5 days a week.

Alternatively, the composition comprising water-insoluble 20(S)-camptothecin compound may be administered to a human patient intramuscularly, transdermally, parenternally, subcutaneously, intraperitoneally, intravenously, intraarterially, sublingually, rectally, transbuccally, intranasally, liposomally, via inhalation, vaginally, intraoccularly, or via local delivery by a catheter.

This method can be used to treat various types of MDS classified according to the French-American-British classification. Preferably, the method may be used to treat the MDS type: refractory anemia (RA), refractory anemia with ringed sideroblasts (RAS), refractory anemia with excess blasts (RAES), or refractory anemia with excess blasts in transformation (RAES-t).

It is to be understood that both the foregoing general description and the following detailed description are exemplary and explanatory only and are intended to provide further explanation of the present invention, as claimed.

### DETAILED DESCRIPTION OF THE PRESENT INVENTION

The present invention provides methods for treating hematologic disorders, in particular, chronic leukemia and the myelodysplastic syndromes using water insoluble 20(S)-camtothecin compounds with a close closed-lactone ring. Inventors believe that 20(S)-camtothecin compounds with a close closed-lactone E-ring is essential for stabilizing the cleavable complex associated with in topoisomerase I (topo I) enzymatic activity, leading to inhibition of topo I function and subsequently cell death. By using a water insoluble camptothecin compound in the treatment, the abnormal hematologic cells potentially having a higher amount of topo I may be more susceptible to inhibitory effects of the camptothecin compound, which may result in less side effects.

1. Method of Treating Chronic Leukemia with Oral Dosage form of Water-Insoluble 20(S)-camptothecin

In one aspect of the present invention, a method is provided for treating treating a hematologic disorder in a patient by using water insoluble 20(S)-camtothecin compound in oral dosage forms. The method comprises: orally administering into the patient suffering from a hematologic disorder a composition that is adapted for oral administration and comprises an effective amount of water-insoluble 20(S)-camptothecin compound with a closed-lactone ring.

This method can be used to treat a hematologic disorder such as various forms of leukemia. Forms or classes of leukemia include, but are not limited to, chronic myeloid leukemia (CML), chronic myelomonocytic leukemia (CMML), acute myeloid leukemia (AML), and acute lymphoblastic leukemia (ALL). In particular, this method can be used to treat a patient with CML, Philadelphia chromosome (Ph)-negative or Ph-positive CML.

By using an oral dosage form of the camptothecin compound, patients with leukemia, especially chronic leukemia, can benefit from such a convenient and less painful route of administration. Since chronic leukemia such as CML and CCML has a relatively indolent course of disease, it is desirable to have a prolonged treatment at lower dose and for a longer exposure schedule. Water-insoluble camptothecin compound, especially 9-nitro-camptothecin (9NC), is stable in a solid form and can be conveniently adapted for oral administration. Patients can take this drug in various oral dosage forms, such as capsules, tablets and gelules at home or in the clinic.

In comparison, common chemotherapeutic agents against leukemia, such as ara-C, is usually administered via bolus IV injection, continuous IV infusion, or intrathecally administration. Topotecan, a water soluble analogue of camptothecin, also requires IV infusion, continuous or over 30-min daily. Such routes of administration compromise the quality of life of the patients and increase the burden of health care management.

The method of the present invention solve these problems associated with IV injection or infusion by providing a more patient-friendly way of treatment. Because the method can be used for a prolonged period of treatment schedule at a lower dose, the treatment regimen may be more efficacious and less toxic than sporadic injection of chemotherapeutic agent at a much higher dosages.

According to this method the water-insoluble 20(S)-camptothecin compound is 20(S)-camptothecin, an analog of 20(S)-camptothecin, or a derivative of 20(S)-camptothecin. The analogs and derivatives of 20(S)-camptothecin also include pharmaceutically active metabolites of 20(S)-camptothecin and prodrugs of 20(S)-camptothecin.

When used in the context of this invention, 20(S)-camptothecin is the plant alkaloid 20(S)-camptothecin. Examples of camptothecin derivatives or analogs include, but are not limited to, 9-nitro-20(S)-camptothecin, 9-20(S)-camptothecin, 9-methyl-camptothecin, 9-chloro-camptothecin, 9-flouro-camptothecin, 7-ethyl camptothecin, 10-methyl-camptothecin, 10-chloro-camptothecin, 10-bromo-camptothecin, 10-fluoro-camptothecin, 9-methoxy-camptothecin, 11-fluoro-camptothecin, 7-ethyl-10-hydroxy camptothecin, 10,11-methylenedioxy camptothecin, and 10,11-ethylenedioxy camptothecin, and 7-(4-methylpiperazinomethylene)-10,11-methylenedioxy camptothecin. Prodrugs of camptothecin include, but are not limited to, esterified camptothecin derivatives as described in U.S. Pat. No. 5,731,316, such as camptothecin 20-O-propionate, camptothecin 20-O-butyrate, camptothecin 20-O-valerate, camptothecin 20-O-heptanoate, camptothecin 20-O-nonanoate, camptothecin 20-O-crotonate, camptothecin 20-O-2′,3′epoxy-butyrate, nitrocamptothecin 20-O-acetate, nitrocamptothecin 20-O-propionate, and nitrocamptothecin 20-O-butyrate.

Native, unsubstituted, camptothecin can be obtained by purification of the natural extract, or may be obtained from the Stehlin Foundation for Cancer Research (Houston, Tex.). Substituted camptothecins can be obtained using methods known in the literature, or can be obtained from commercial suppliers. For example, 9-nitrocamptothecin may be obtained from SuperGen, Inc. (San Ramon, Calif.), and 9-aminocamptothecin may be obtained from Idec Pharmaceuticals (San Diego, Calif.). Camptothecin and various of its analogs may also be obtained from standard fine chemical supply sources, such as Sigma Chemicals.

In a preferred embodiment, the water-insoluble 20(S)-camptothecin compound is 9-nitro-20(S)-camptothecin, 9-amino-20(S)-camptothecin, or combinations thereof.

Also according to this method, the water-insoluble 20(S)-camptothecin compound is administered orally to a patient at an effective amount preferably from about 0.5 mg/m² to about 5.0 mg/m² of body surface area of the patient daily, more preferably from about 1.0 mg/m² to about 3.0 mg/m² of body surface area of the patient daily, and most preferably from about 1.5 mg/m² to about 2.5 mg/m² of body surface area of the patient daily.

Also according to this method, the water-insoluble 20(S)-camptothecin compound is administered orally to a patient at an effective amount daily for preferably 3-6 days a week and more preferably 4-5 days a week.

2. Method of Treating the Myelodysplastic Syndromes with Water-Insoluble 20(S)-camptothecin

The myelodysplastic syndromes are heterogeneous clonal hematopoietic stem cell disorders, including these types: (1) refractory anemia (RA); (2) refractory anemia with ringed sideroblasts (RAS); (3) refractory anemia with excess blasts (RAEB); and (4) refractory anemia with excess blasts in transformation (RAEB-t). The types have different degrees of disordered hematopoiesis, frequencies of transformation to acute leukemia, and prognoses.

In an RA patient, the myeloid and megakaryocytic series in the bone marrow appear normal, but megaloblatoid erythroid hyperplasia is present. Dysplasia is usually minimal. Macrocytic anemia with reticulocytopenia is present in the blood. Transformation to acute leukemia is rare, and median survival varies from 2 to 5 years in most series. This type accounts for 20% yo 30% of patients.

In an RAS patient, the blood and marrow are identical to those in patients with RA, except that at least 15% of marrow red cell precursors are ringed sideroblasts. Only 2% to 5% of patients present with this type, and prognosis is identical to that of RA.

In an RAEB patient, there is significant evidence of disordered myelopoiesis, and megakaryocytopoiesis in addition to abnormal erythropoiesis. The marrow contains 5% to 20% myeloid blasts, and 1% to 5% of blasts may circulate in the blood. Progression to acute leukemia occurs in approximately 40% of patients, and median survival is usually 6 to 9 months. Approximately one-third of patients present with this type.

In an RAEB-t patient, there is a panmyelosis in which 20% to 30% of marrow cells are blasts and more than 5% blasts are seen in the blood. Auer rods may be seen. Sixty to 75% of patients develop overt acute leukemia, and median survival is 6 months or less. Approximately 25% of patients present with this type.

The mainstay of treatment of the myelodysplastic syndromes (MDS) is supportive care. The treat anemia associated with MDS, various methods have been used, including red cell transfusions, use of erythropoietin, granulocyte colony-stimulating factor, or Interferon-alfa, iron chelation therapy with subcutaneously administered desferrioxamine and vitamin C. Results from using chemotherapy with cytotoxic agents (e.g. low-dose cytarabine) are usually disappointing, and responses are often brief when achieved. Low doses of oral melphalan have been reported to have a similar response rate to low-dose cytarabine in small trials, however, the long-term consequences of ongoing alkylator therapy in this patient population are unknown and potentially harmful. Omoto, E. et al. (1996) Low-dose melphalan for treatment of high-risk myelodysplastic syndromes. Leukemia 10(4): 609-614.

The present invention provides a novel method for treating the myelodysplastic syndromes by using water-insoluble compound. The method comprises administering to the patient an effective amount of a water-insoluble 20(S)-camptothecin compound with a closed-lactone ring, a derivative thereof, or a mixture thereof.

Preferably, the 20(S)-camptothecin compound is at least one compound selected from 20(S)-camptothecin, 9-nitro-20(S)-camptothecin, and 9-amino-20(S)-camptothecin. According to a preferred embodiment of the present invention an effective amount of 9-nitro-20(S)-camptothecin is used to treat a patient afflicted with a disorder of MDS.

The 20(S)-camptothecin compound with a closed lactone ring can be administered via any of a variety of routes. Preferably the compound is administered orally, intramuscularly, or transdermally, although the compound can instead be administered via any parenternal or subcutaneous route or through an inhaler or other airborne delivery systems and the like.

According to the present invention, the inventors discovered that the water-insoluble 20(S)-camptothecin compound, in particular, 9NC, have been successful in providing a safe and effective treatment for patients suffering from MDS. According to the present invention, the use of 9-Nitro-20(S)-camptothecin is preferred in the treatment of MDS.

The compounds used according to the present invention are not particularly limited in form with respect to how the compound is administered to a patient. Pharmaceutical compositions containing the compound can include pharmaceutically acceptable diluents, vehicles, carriers, and the like. Pharmaceutical compositions for use in accordance with the present invention can also include other active ingredients. Exemplary forms of administration include, for example, orally administered forms such as tablets, powder, capsules, and syrups; and parenternal administration forms such as liquid injections, for example, solutions, emulsions, suspensions, and fat emulsions, and external preparations such as suppositories, inhalables, liniments, and aerosols. The preparations of these described administration forms and others can be carried out by conventional means. Methods of forming the administration forms can include the blending of compound as an active ingredient with pharmaceutically necessary additives such as excipients, binders, disintegrates, lubricants, diluents, and isotonic agents. Further, if desired, other additives may be included in the administered forms of the compound, including flavorings, colorants, perfumes, microbicides, antiseptics, and stabilizers.

The amount of compound and the time of administration of the compound according to the present invention may vary depending upon symptoms, age, body weight, and administration form. The number of daily treatments per week may vary based on many factors and preferably ranges preferably from 3 to 6 consecutive daily treatments per week and more preferably from 4 to 5 consecutive daily treatments per week.

When treating humans, the dosage is preferably calculated based on body surface area. The effective amount can be from about 0.5 mg/m² of body surface area daily to about 5.0 mg/m² of body surface area daily, preferably for five days per week. More preferably, the effective amount is from about 1.0 mg/m² of body surface area daily to about 3.0 mg/m² of body surface area daily five days per week. Most preferably, the effective amount is from about 1.5 mg/m² of body surface area daily to about 2.5 mg/m² of body surface area daily five days per week.

9-Nitro-20(S)-camptothecin is preferably provided in an oral dosage containing from about 0.25 mg to about 1.0 mg encapsulated active compound.

Toxicology studies performed in mice and dogs demonstrated a primary toxicity that was intestinal. In dogs, dosages of 1.0 mg/kg/day were well tolerated even when administered for over 2 months.

Depending upon what former treatments or current treatments a patient is undergoing, the maximum tolerable dosage can vary. Preferably, the maximum dosage will not exceed about 3.0 mg/m² of body surface area daily during a regiment consisting of 5 consecutive days of treatment followed by two consecutive days without treatment.

In patients that have been heavily pre-treated with chemotherapy drugs, the starting dosage is preferably lowered to about 1.5 mg/m² of body surface area on a regiment consisting of 5 consecutive days of treatment followed by two consecutive days without treatment. A course of treatment involves 4 to 6 weeks of treatment following these regiments.

According to the present invention, water-insoluble 20(S)-camptothecin compounds with a closed lactone ring have been found to stabilize topo I DNA complexes during the enzyme's strand passing action thus inhibiting topo I function. The hematopoietic lineages from the abnormal stems cells may be more susceptible to camptothecin compound with a closed lactone ring than normal cells. Thus, using water-insoluble camptothecin compound with a closed a lactone ring should be safer and has fewer side effects than conventional chemotherapeutic agents such as ara-C and alkylators.

The present invention may be useful for patients suffering from MDS but is preferably reserved for patients over 15 years of age. Preferably, the patients have been off of chemotherapy for at least 2 weeks prior to entering a treatment regiment according to the present invention. More preferably, the patient has recovered from all toxicity associated with any previous chemotherapy treatment. Preferably, the patient has an adequate liver function determined by a bilirubin count of less than or equal to 1.5 mg %, and an adequate renal function determined by a creatinine level of less than or equal to 2 mg %. Preferably, the patient is not a nursing female and will not be conceiving a child during the course of the treatment.

Courses of treatment can be repeated every 4 to 6 weeks depending upon patient response and recovery of blood cell and platelet counts. For patients exhibiting low whole blood cell counts due to myelosuppresion, and not due to disease, a subsequent course of treatment should not begin until the whole blood cell count exceeds 2.0×10⁹/L and until the platelet counts exceeds 80×10⁹/L. For patients having low whole blood cell counts and platelet counts attributable to persistent disease, subsequent courses of treatment can be started regardless of the counts.

During the course of treatment, dose modifications are preferably reserved for different courses of treatment as opposed to changes of daily dosages within a single course. Depending upon patient response, the daily orally administered amount of 9-nitro-20(S)-camptothecin is increased or decreased by 0.5 mg/m² per course. Preferably, the minimum treatment amount is no lower than 1.5 mg/m² administered orally daily for four consecutive days per week followed by 3 consecutive days with no treatment, each week.

The daily dosage, regardless of the amount, can be given in one or more portions, for example, in two equally divided oral dosages given, for example, at 8:00 AM in the morning and at 8:00 PM at night.

Preferably, an evaluation of the efficacy of the treatment should not be definitively concluded until a patient receives a minimum of 2 courses of treatment. After a minimum of 2 courses, an evaluation of the trial can be made. Grade III or IV hemotologic toxicity during remission induction should not prompt dose changes. Preferably, only prolonged myelosuppresion defined as a hypocellular marrow (less than 5% cellularity) 6 weeks from the start of therapy would require dose reduction in the form of a 0.5 mg/m² daily reduction in dose. A maximum of twelve courses of treatment is preferred.

Herein, the phrase “complete remission” refers to a normalization of the peripheral blood and bone marrow with 5% or less blasts, normal or hypercellular marrow, and a granulocyte count of 10⁹/L or above, with each of these conditions lasting for at least 4 weeks. Complete remission does not require the disappearances of the abnormal karyotype. Herein the phrase “partial remission” refers to an improvement of at least 2 of the following parameters (1) platelet increased by 100% and to above 50×10⁹/L if it was below that level, (2) granulocyte increase by 100% and to about 10⁹/L if it was below that level, (3) a hemoglobulin increase by 2 g/dl if it was below 10 g/dl, or (4) a reduction of marrow blasts to 5% or less if it was 10% or more. To be considered in partial remission, at least two of these parameters should show the improvement and the improvement should last for at least four weeks.

The present invention will be further clarified by the following examples, which are intended to be exemplary of the present invention.

### EXAMPLE

1. Drug Product

9-nitro-20(S)-camptothecin (9NC) was encapsulated in oral dosage of gelules containing 1 mg 9NC by a registered pharmacist. When 9NC is left at room temperature (25° C.) for 2 months, the loss of activity is minimal and has been estimated to be less than 5%+4.6%. Determination of activity has been done by HPLC. The gelules are preferably stored in freezers.

2. Clinical Treatment of Hematologic Malignancies with 9NC

Twenty five patients with hematologic disorders were admitted for the treatment with 9NC. Among them, 9 patients had Philadelphia chromosome (Ph)-negative bcr/abl-negative CML; 8 patients had Philadelphia chromosome (Ph)-positive CML (6 in chronic phase and 2 in accelerated phase); 7 patients had CMML; and one had MDS (RAEB, refractory anemia with excess blasts). Median age of patients was 65 years, ranging from age 29 to age 77. Median white blood cell (WBC) counts was 25.2×10⁹/L (range: 4.5-76×10⁹/L); and median platelet count was 223×10⁹/L (range: 23-1042×10⁹/L). Among the patients for the treatment, 14 patients had splenomegaly, with median of 10.5 cms below costal margin (range: 1-13 cms). The 8 patients with Ph-positive CML were previously treated with interferon (IFN). Except for 5 patients, 12 other patients were also previously treated with IFN. Prior to the treatment of the present invention, the patients were off chemotherapy using other anticancer drugs for 2 weeks and recovered from toxicity of that chemotherapy.

The dosing schedule was designed as the following. The drug product, 9NC gelules, are orally administered into patients for 5 consecutive days every week. Four weeks of treatment with 9NC oral dosage defines one course of treatment. Courses are repeated every 4-6 weeks, depending on patient response and recovery of WBC and platelet counts. The total daily 9NC dose is given in 2 equal divided doses (e.g. 8 a.m. and 8 p.m.).

The dose of treatment with 9NC was designed to be modified at 5 dose levels according to myelosuppression and extramedullary toxicity. The starting dose schedule at level 0 is 2.0 mg/m² 9NC orally daily, 5 days every week. Dose level +1 is 2.5 mg/m² 9NC orally daily, 5 days every week. Dose level +2 is 3.0 mg/m² 9NC orally daily, 5 days every week. Dose level −1 is 1.5 mg/m² 9NC orally daily, 5 days every week. Dose level −2 is 1.5 mg/m² 9NC orally daily, 4 days every week.

During the course of treatment, if there is no response to 9NC and the patient's hematologic toxicity is less than grade II, the dose level is increase by 1 level. If there is no response to 9NC and the patient's hematologic toxicity is at grade II, the dose level stays at the same level. If there is response to 9NC and the patient's hematologic toxicity is at grade III or IV, the dose level is decreased by 1 level. If there is no response to 9NC and the patient's hematologic toxicity is at grade III or IV, the patient is off the treatment with 9NC.

If low hematologic counts is due to myelosuppression, not disease, subsequent courses can be delayed until WBC >2.0×10⁹/L and platelets >80×10⁹/L. If low counts are attributed to persistent disease, subsequent courses are started regardless of the counts.

During remission induction, grade III or IV hematologic toxicity does not prompt dose changes. Only prolonged myelosuppression defined as a hypocellular marrow (<5% cellularity) 6 weeks from start of therapy would require dose reduction by decreasing 1 dose level.

i) Treatment of CML Patients with 9NC

The criteria for response to patients with CML are as follows.

Complete hematologic remission (CHR): Normalization for at least 4 weeks of the bone marrow (less than 5% blasts) and peripheral blood with WBC <10×10⁹/L and no peripheral blasts, promyelocytes or myelocytes. This is in addition to disappearance of all signs and symptoms of the disease.

Partial hematologic remission (PHR): CHR except for persistence of immature cells (myelocytes, metamyelocytes), or splenomegaly <50% of pretreatment, or thrombocytosis >450×10⁹/L but <50% of pretreatment.

For patients with Ph-positive CML, complete hematologic remission is further classified according to suppression of the Philadelphia chromosome (Ph) as: no cytogenetic response (Ph positive 100%), minimal cytogenetic response (Ph positive 34-90%), partial cytogenetic response (Ph positive 1-34%), and complete cytogenetic response (Ph positive 0%).

a) Ph-Negative bcr/abl-Negative CML

Gelules of 9NC were orally administrated into the 9 patients with Ph-negative bcr/abl-negative CML. The starting dose was 2 mg/m² daily for 5 days every week. Doses were adjusted according to myelosuppression and extramedullary toxicity as described above. Based on the criteria set forth above for CML, among these 9 patients, 2 patients had complete remission; 4 patients had partial remission; 1 patient was stable; and 2 patients were resistant to the treatment. Thus, the objective response (OR) was 6/9. In all 8 patients with splenomegaly there was at least minor spleen response. These results demonstrate that orally-administered 9NC has significant activity in Ph-negative CML.

b) Ph-Positive CML

Gelules of 9NC were orally administrated into the 8 patients with Ph-positive CML. The starting dose was 2 mg/m² daily for 5 days every week. Doses were adjusted according to myelosuppression and extramedullary toxicity as described above. Based on the criteria set forth above for CML, among these 8 patients, 1 patient had complete response hematologically and cytogenetically; and 1 patient in accelerated phase had minor spleen response. The other 6 patients did not show response to the treatment. These results demonstrate that orally-administered 9NC can induce cytogenetical response in patients with Ph-positive CML, i.e. reduction of the Philadelphia chromosomes in the patients' blood cells.

ii) Treatment of CMML Patients with 9NC

Gelules of 9NC were orally administrated into the 7 patients with CMML. The starting dose was 2 mg/m² daily for 5 days every week. Doses were adjusted according to myelosuppression and extramedullary toxicity as described above. Based on the criteria set forth above for CML, among these 7 patients, 2 patients had partial remission; 4 patients had hematologic improvement in WBC or splenomegaly; and 1 patient was resistant to the treatment. Thus, the objective response (OR) was 6/7. These results demonstrate that orally-administered 9NC has significant activity in CMML.

3. Treatment of MDS Patient with 9NC

The criteria for response to patients with MDS are as follows.

Complete remission (CR): Normalization of the peripheral blood and bone marrow 5% or less blasts, normo- or hypercellular marrow, a granulocyte court of 10⁹/L or above, lasting for at least 4 weeks. CR does not require disappearance of the abnormal karyotype although this is evaluated at the time of CR.

Partial remission (PR): Improvement of at least two of the following parameters: 1) platelet increase by 100% and to above 50×10⁹/L if it was below that level; 2) granulocyte increases by 100% and to above 10⁹/L if it was below that level; 3) hemoglobin increase by 2 g/dl if it was below 10 g/dl; or 4) reduction of marrow blasts to 5% or less if it was 10%. This should last for at least 4 week.

Gelules of 9NC were orally administrated into the patient with MDS of the RAEB type. The starting dose was 2 mg/m² daily for 5 days every week. Doses were adjusted according to myelosuppression and extramedullary toxicity as described above. Based on the criteria set forth above for MDS, this patient with MDS had complete remission. This result demonstrate that orally-administered 9NC was highly active in this patient with MDS and caused normalization of peripheral bloods and bone marrow

In summary, treatments using 9-nitro-20(S)-camptothecin in oral dosage have proven successful in treating patients suffering from hematologic disorders, especially in patients with CML and MDS. This drug can be taken orally at low dosages over a long exposure schedule. Accordingly, 9NC, as well as other camptothecin analogs and derivatives, are promising in the treatment of various forms of leukemia, MDS and other forms of myoloproliferative disorders via oral administration. Such a route of administration is much more convenient and less painful than infusion of water-soluble chemotherapeutic agents into the patient, thereby improving quality of life of the patient.

Other embodiments of the present invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for treating a hematologic disorder in a patient, comprising:
orally administering into the patient suffering from a hematologic disorder a composition that is adapted for oral administration and comprises
an effective amount of water-insoluble 20(S)-camptothecin compound with a closed-lactone ring.

2. The method of claim 1, wherein the water-insoluble 20(S)-camptothecin compound is 20(S)-camptothecin, an analog of 20(S)-camptothecin, or a derivative of 20(S)-camptothecin.

3. The method of claim 1, wherein the water-insoluble 20(S)-camptothecin compound is 9-nitro-20(S)-camptothecin, 9-amino-20(S)-camptothecin, or combinations thereof.

3. The method of claim 1, wherein the water-insoluble 20(S)-camptothecin compound is administered orally to a patient at an effective amount from about 0.5 mg/m² to about 5.0 mg/m² of body surface area of the patient daily.

4. The method of claim 1, wherein the water-insoluble 20(S)-camptothecin compound is administered orally to a patient at an effective amount from about 1.0 mg/m² to about 3.0 mg/m² of body surface area of the patient daily.

5. The method of claim 1, wherein the water-insoluble 20(S)-camptothecin compound is administered orally to a patient at an effective amount from about 1.5 mg/m² to about 2.5 mg/m² of body surface area of the patient daily.

6. The method of claim 1, wherein the water-insoluble 20(S)-camptothecin compound is administered orally to a patient at an effective amount daily for 3-6 days a week.

7. The method of claim 1, wherein the water-insoluble 20(S)-camptothecin compound is administered orally to a patient at an effective amount daily for 4-5 days a week.

8. The method of claim 1, wherein the hematologic disorder is leukemia.

9. The method of claim 8, wherein the leukemia is chronic myeloid leukemia, chronic myelomonocytic leukemia, acute myeloid leukemia, or acute lymphoblastic leukemia.

10. The method of claim 7, wherein the chronic myeloid leukemia is Philadelphia chromosome-negative chronic myeloid leukemia.

11. A method of treating a subject afflicted with myelodysplastic syndrome, comprising:
administering to said patient a composition comprising an effective amount of at least one water-insoluble 20(S)-camptothecin compound of the closed lactone ring form, a derivative thereof, or a mixture thereof.

12. The method of claim 11, comprising: administering 9-nitro-20(S)-camptothecin or a mixture of 9-nitro-20(S)-camptothecin with a 20(S)-camptothecin compound with a closed lactone ring or a derivative thereof.

13. The method of claim 11, wherein the treated subject is a human patient.

14. The method of claim 11, wherein the composition is orally administered into a human patient.

15. The method of claim 14, wherein the effective amount is from about 0.5 mg/m² to about 5.0 mg/m² of body surface area of the patient daily.

16. The method of claim 14, wherein the effective amount is from about 1.0 mg/m² to about 3.0 mg/m² of body surface area of the patient daily.

17. The method of claim 14, wherein the effective amount is from about 1.5 mg/m² to about 2.5 mg/m² of body surface area of the patient daily.

18. The method of claim 14, wherein the water-insoluble 20(S)-camptothecin compound is administered orally to a patient at an effective amount daily for 3-6 days a week.

19. The method of claim 14, wherein the water-insoluble 20(S)-camptothecin compound is administered orally to a patient at an effective amount daily for 4-5 days a week.

20. The method of claim 11, wherein the composition is administered to a human patient intramuscularly or transdermally.

21. The method of claim 11, wherein the composition is administered to a human patient parenternally, subcutaneously, intraperitoneally, intravenously, intraarterially, sublingually, rectally, transbuccally, intranasally, liposomally, via inhalation, vaginally, intraoccularly, or via local delivery by a catheter.

22. The method of claim 11, where the myelodysplastic syndrome is of the type refractory anemia, refractory anemia with ringed sideroblasts, refractory anemia with excess blasts, or refractory anemia with excess blasts in transformation.

23. The method of claim 11, where the myelodysplastic syndrome is of the type refractory anemia with excess blasts.
